(19) United States
(12) Patent Application Publication
(10) Pub. No.: US 2009/0216268 A1
(43) Pub. Date: Aug. 27, 2009

(54) SUTURE AND SUTURING TECHNIQUE FOR FACILITATING KNOTTING

(76) Inventor: Gideon G. Panter, New York, NY (US)

Correspondence Address: DR. MARK M. FRIEDMAN;C/O BILL POLKINGHORN - DISCOVERY DISPATCH, 9003 FLORIN WAY, UPPER MARLBORO, MD, 20772, US

(21) Appl. No.: 12/034,693

(22) Filed: Feb. 21, 2008

(51) Int. Cl. A61B 17/06; A61B 17/04

(52) U.S. Cl. 606/222; 606/228

(57) ABSTRACT

A suture for facilitating tying of knots includes a length of suture material fixed to a needle. At least part of the length of the suture material is resiliently biased to a coiled state which is straightenable under applied tension but which returns to its coiled state when released. In use, part of the suture material is drawn through a region of tissue so that portions of the suture material lie on opposite sides of the tissue. A knot is then formed in the suture material by passing one of the portions through at least one resiliently-formed coil formed in the other of the portions, and pulling tight the suture material.

### FIELD AND BACKGROUND OF THE INVENTION

The present invention relates to surgery and, in particular, it concerns a method for suturing and a corresponding suture structure which facilitates the process of forming knots.

A routine part of most surgical procedures is suturing, also referred to as “sewing” or “stitching”, in which two regions of tissue, for example, edges of an incision or wound to be fastened together, are connected using a thread-like element referred to as a “suture”. While many alternative forms of fastening have been proposed, and some have entered common usage, the most common form of fastening remains the use of a suture which is tied by the surgeon to form a knot. A typical surgeon's knot includes at least two throws (e.g., a double or triple knot) in which at least the first throw has the ends wound together twice. One example of such a knot is illustrated in FIG. 1.

Tying of surgical knots is a skilled task, and can sometimes take a significant proportion of the total time of a surgical procedure. This is particularly true where a series of “interrupted” or individual sutures are required, each requiring its own knot.

Tying of sutures is particularly challenging in the context of minimally invasive surgical techniques where the tying is performed within the body by use of tools manipulated from outside the body (or remotely controlled). An example of a sequence used for forming intra-corporeal knots is illustrated in FIG. 2, steps (a) through (d). After insertion of the suture material through the tissue to be sutured, a first gripping tool 10 is used to grip a first portion of the suture material and to wind it a number of times around a second gripping tool 12 as shown in (a). Second gripping tool 12 is then used to grip the other end of the suture material and draw it back through the loops as shown in (b). The two gripping tools are then pulled apart to arrange and tighten the first throw of the knot, as shown in (c) and (d). The process is then repeated, again winding the one side of the suture material around second gripping tool 12 and drawing the other end through the loops, until the desired number of throws have been formed.

While this procedure may not appear particularly complicated, the process of winding suture material around one gripping tool by use of another gripping tool, both manipulated from outside the body, is in practice difficult to perform, requiring much practice and frequently leading to delay and frustration as the loops repeatedly slip off the tool before the knot is completed. Even in the hands of an experienced surgeon, tying of sutures in a minimally invasive procedure may account for a major part of the total duration of the procedure.

There is therefore a need for a method and corresponding suture which would facilitate and render more efficient the process of tying sutures.

### SUMMARY OF THE INVENTION

The present invention is a suture and a corresponding method for suturing.

According to the teachings of the present invention there is provided, a method for suturing tissue comprising: (a) providing a length of suture material having a leading end and a trailing end, at least part of the length being resiliently biased to a coiled state, the at least part of the length being straightenable under tension and returning to the coiled state when released; (b) drawing the leading end through or around at least one region of tissue so that a leading portion of the suture material lies between the tissue and the leading end and a trailing portion of the suture material lies between the tissue and the trailing end; and (c) forming a knot in the suture material, the forming a knot including at least one sequence of: (i) passing at least part of one of the leading and trailing portions through at least one resiliently-formed coil formed in the other of the leading and trailing portions, and (ii) pulling tight the leading and trailing portions.

According to a further feature of the present invention, the passing is performed by inserting a gripping tool through the at least one resiliently formed coil, gripping the other of the leading and trailing portions, and drawing the other of the leading and trailing portions back through the at least one resiliently formed coil.

According to a further feature of the present invention, the gripping tool is inserted through the at least one resiliently formed coil from proximal to distal, and the drawing back through the at least one resiliently formed coil is from distal to proximal.

According to a further feature of the present invention, the passing is performed so as to pass the entirety of the one of the leading and trailing portions through the at least one resiliently-formed coil.

According to a further feature of the present invention, the passing is performed so as to pass the at least part of the one of the leading and trailing portions through at least two resiliently-formed coils formed in the other of the leading and trailing portions.

According to a further feature of the present invention, the passing is performed so as to pass the trailing portion through the at least one resiliently-formed coil formed in the leading portion.

According to a further feature of the present invention, the sequence of passing and pulling tight is performed at least three times.

According to a further feature of the present invention, a remainder of the leading portion is severed from the knot and steps (a) through (c) are repeated to form a subsequent knot.

According to a further feature of the present invention, the leading end of the suture material is fixed to, and aligned as a continuation of, a needle.

According to a further feature of the present invention, a major part of the length of the suture material is resiliently biased to the coiled state.

According to a further feature of the present invention, the coiled state approximates to a helical form.

According to a further feature of the present invention, the drawing and forming a knot are performed within a body cavity by manipulation of tools held outside the body.

According to a further feature of the present invention, the drawing and forming a knot are performed by robotically controlled tools.

There is also provided according to the teachings of the present invention, a suture for facilitating tying of knots, the suture comprising: (a) a needle; and (b) a length of suture material fixed to the needle, wherein at least part of the length of suture material is resiliently biased to a coiled state, the at least part of the length being straightenable under tension and returning to the coiled state when released.

According to a further feature of the present invention, a major part of the length of the suture material is resiliently biased to the coiled state.

According to a further feature of the present invention, the coiled state approximates to a helical form.

According to a further feature of the present invention, the coiled state includes a plurality of coils having an external diameter of between about 3 and about 30 millimeters.

According to a further feature of the present invention, the coiled state includes a plurality of coils having an external diameter of between about 10 and about 20 millimeters.

According to a further feature of the present invention, the suture material is a biodegradable material.

According to a further feature of the present invention, the suture material is a braided material including at least one resilient strand configured to provide the resilient bias.

According to a further feature of the present invention, the suture material includes a resilient core configured to provide the resilient bias.

According to a further feature of the present invention, the suture material has a coating configured to provide the resilient bias.

There is also provided according to the teachings of the present invention, a method for producing a suture for facilitating tying of knots, the method comprising, (a) forming a length of material into a coiled form including a plurality of coils; and (b) processing the length of material so as to impart to the length of material an elastic memory such that the length of material is resiliently biased to the coiled state, being straightenable under applied tension and returning to the coiled state when released.

According to a further feature of the present invention, the processing includes heat treating the length of material.

According to a further feature of the present invention, the processing includes applying a coating to the length of material.

According to a further feature of the present invention, a needle is fixed at one end of the length of material.

### BRIEF DESCRIPTION OF THE DRAWINGS

The invention is herein described, by way of example only, with reference to the accompanying drawings, wherein:

FIG. 1 is a schematic example of a conventional surgeon's knot, described above;

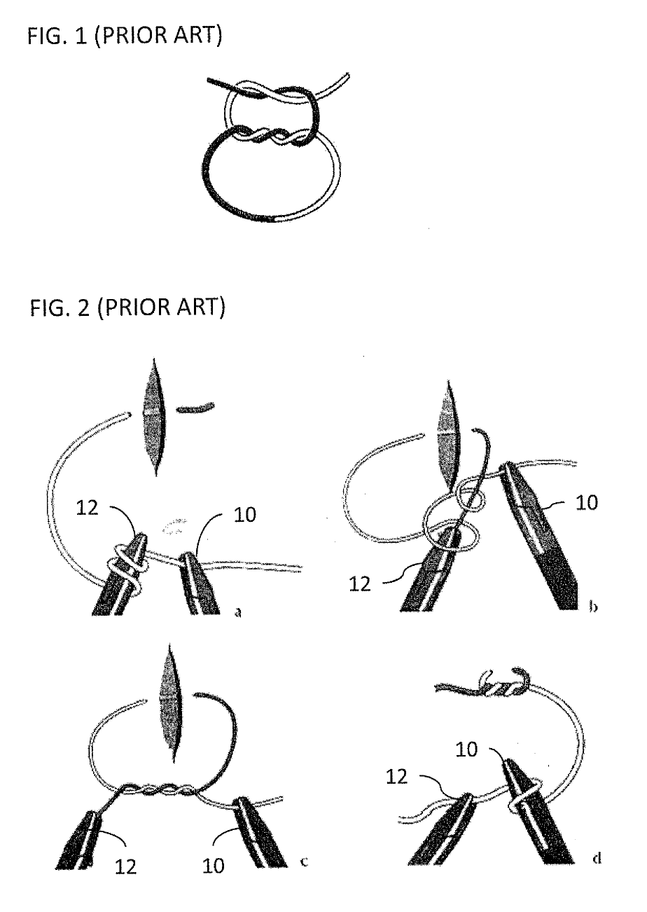

FIG. 2 is a schematic representation of four stages in a conventional process for tying an intra-corporeal surgeon's knot, described above;

FIG. 3 is a schematic isometric view of a suture, constructed and operative according to the teachings of the present invention;

FIGS. 4A-4C are schematic representations of a sequence of tying a suture according to the teachings of the present invention;

FIGS. 4D and 4E are schematic representations of variants of the sequence of FIGS. 4A-4C; and

FIGS. 5 and 6 are schematic isometric views of two alternative implementations of a suture, constructed and operative according to the teachings of the present invention.

### DESCRIPTION OF THE PREFERRED EMBODIMENTS

The present invention is a suture and a corresponding method for suturing.

The principles and operation of sutures and methods for suturing according to the present invention may be better understood with reference to the drawings and the accompanying description.

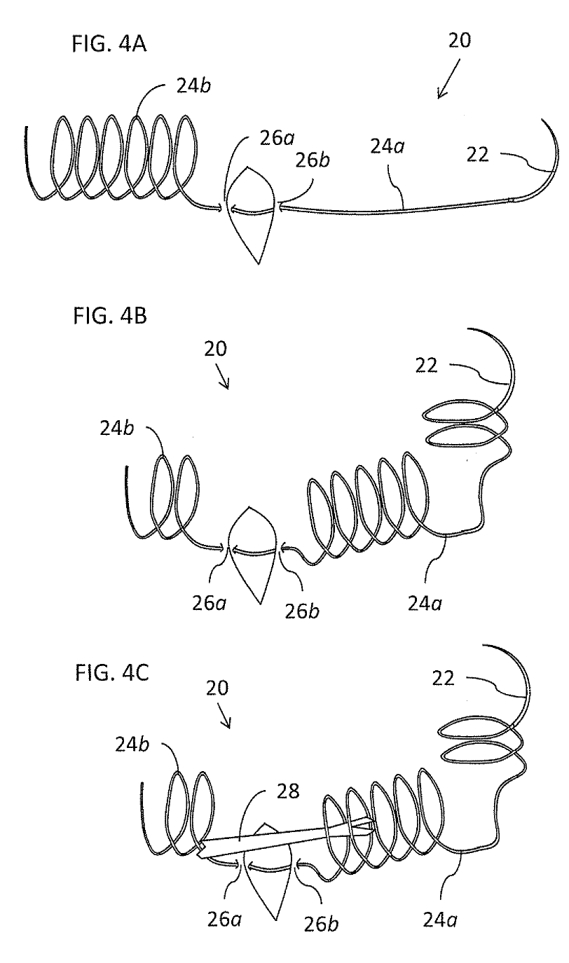

Referring now to the drawings, FIG. 3 illustrates a suture, generally designated 20, constructed and operative according to the teachings of the present invention, for facilitating tying of knots. Suture 20 includes a needle 22 to which is attached a length of suture material 24. It is a particular feature of the present invention that at least part of the length of suture material 24 is resiliently biased to a coiled state in such a manner that it is straightenable under applied tension and yet returns to its coiled state when released.

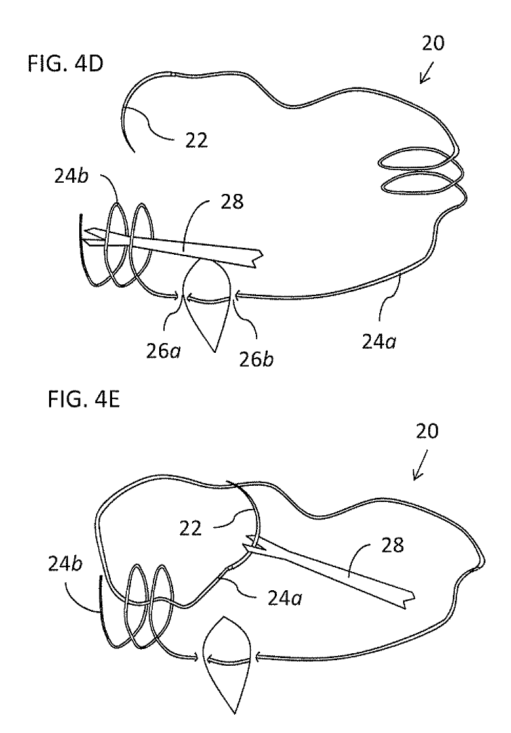

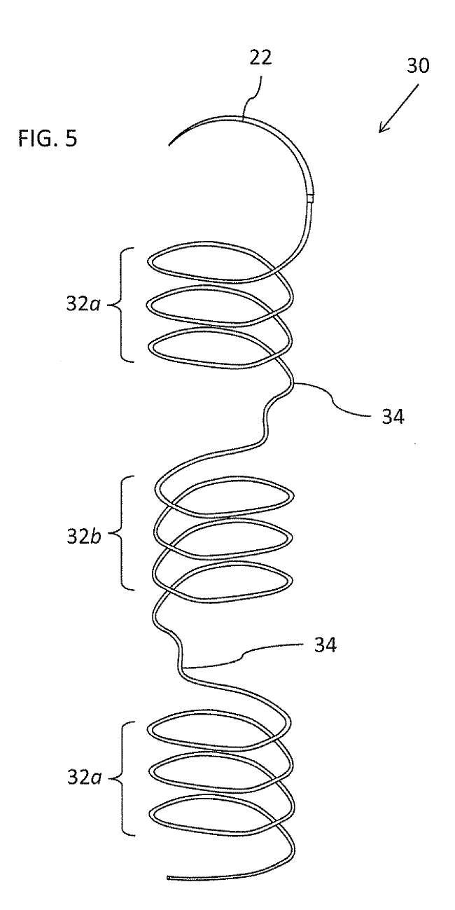

The significance of this structure will be understood with reference to FIGS. 4A-4E which illustrate a method of suturing according to the present invention. Specifically, the leading end of suture material, led by needle 22, is drawn through (or around) at least one region of tissue 26a, 26b so that a leading portion 24a of the suture material lies between the tissue and the leading end and a trailing portion 24b of the suture material lies between the tissue and the trailing end. Tension applied to needle 22 as the suture material is drawn through the tissue is effective to temporarily straighten the suture material as shown so that it passes easily through the tissue, as seen in FIG. 4A. The coiled form at least partially recovers when this tension is released, as seen in FIG. 4B. A knot is then formed in suture material 24 by passing at least part of one of the leading and trailing portions 24a, 24b through at least one resiliently-formed coil formed in the other of the leading and trailing portions 24a, 24b, and tightening the two portions to form one throw of a knot. This process is repeated however many times is required to form a desired knot.

Since the coils are self-forming under the resilient bias of suture material 24, there is no need for the surgeon to go through a procedure of winding the suture material about a tool. Instead, he or she may simply insert a gripping tool 28 through one or more of the coils and then draw the other portion of the suture material back through the coils, or alternatively, pass needle 22 directly through one or more of the coils of the trailing portion 24b.

It will already be apparent that the resiliently biased coiled state of the suture material thus provides profound advantages for tying sutures. Specifically, instead of the awkward winding action required for tying a conventional straight suture, the pre-biased coiled state of the suture material provides pre-formed loops through which an end of the suture material can be inserted. This and other advantages of the present invention will be better understood with reference to the following detailed description.

Before addressing the features of the present invention in more detail, it will be helpful to define certain terminology as used herein in the description and claims. Firstly, the term “suture material” is used to refer to any flexible elongated element which is capable of being tied into a knot. The term includes any and all types of suture material commercially available, or which may become available in the future, including, but not limited to, natural and synthetic sutures, absorbable and non-absorbable sutures, monofilament and braided sutures, with or without coatings. Some non-limiting examples of raw materials for sutures include, natural materials such as catgut or reconstituted collagen, cotton, silk, and linen; synthetic absorbable materials such as DLPLA (poly(d1-lactide)), LPLA (poly(1-lactide)), PGA (polyglycolide), PDO (poly(dioxanone)), PGA-TMC (poly(glycolide-co-trimethylene carbonate)), PGA-LPLA (poly(1-lactide-co-glycolide)), PGA-DLPLA (poly(d1-lactide-co-glycolide)), LPLA-DLPLA (poly(1-lactide-co-d1-lactide)) or PDO-PGA-TMC (poly(glycolide-co-trimethylene carbonate-codioxanone)); and synthetic nonabsorbable materials such as polypropylene, polyester, polyethylene terephthalate, polybutylene terephthalate, polyamide, various nylons, Goretex® and metallic materials such as stainless steel. The suture material may be packaged pre-sterilized or may be provided ready for sterilization prior to use. The term “suture” is used to refer to a length of suture material ready for use, and typically, refers to the combination of such a length of suture material integrated with a needle so as to be ready for use. However, it will be appreciated that sutures without a needle are also of use according to the teachings of the present invention, for example, for tying and cinching about a grasped elevated portion of tissue, so as to compress the grasped tissue when knotted, thereby closing a bleeding blood vessel or a space. In order to avoid confusion, the final result of a tied portion of suture material connecting together two regions of tissue (which is often also referred to as a “suture”), or tied around a single region of tissue, will be referred to as a “stitch”.

When a portion of suture material is referred to as “between” the tissue and either the leading or trailing end of the suture material, this refers to relation along the length of the suture material itself. Clearly, since the suture material is flexible, the portion “between” two regions of the material is not necessarily spatially between the two regions.

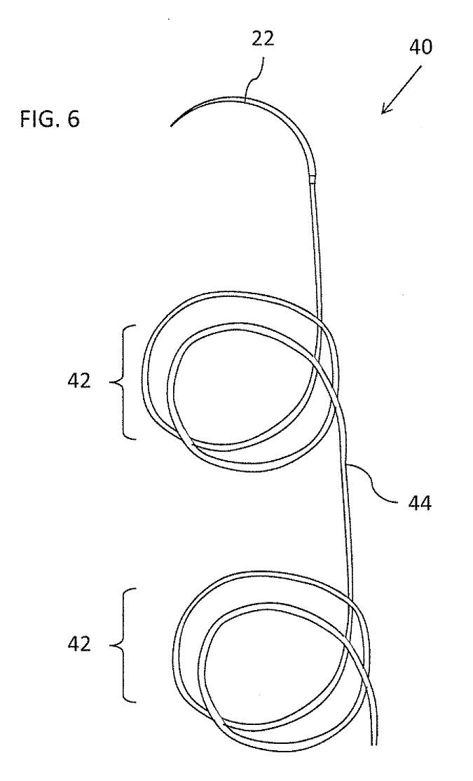

The phrase “coiled state” is used herein in the description and claims to refer to any three-dimensional form which readily allows catching of two or more loops around a straight tool without requiring a rotary motion of either the tool or the suture material. The “coils” or “loops” need not be circular, and need not be continuous along the length of the suture material. For example, the “coiled state” may have isolated coils or groups of coils separated by straight or otherwise shaped portions, or separated by portions which are not resiliently biased to any particular form. Most preferably, the “coiled state” includes one or more group of coils with a roughly helical form. Clearly, when referring to the form of a flexible suture, the form will vary somewhat from a true geometrical helix, due for example to sagging of the suture material under its own weight, non-uniformities in the material or any other mechanical forces acting on the material. In certain implementations (such as will be described with reference to FIG. 5, below), groups of coils may have differing handedness, thereby avoiding a net twisting effect when the suture material is straightened under tension or re-forms the coils when tension is released.

Unless otherwise specified, the term “coil” itself is used herein to refer to individual turns of the coiled state. A portion of suture material is considered a “coil” or “loop” if an element passing through the loop would be caught by the suture material if two points on the loop were pulled directly apart. Similarly, another portion of the suture material is considered “passing through” a coil if the coil would catch or become wound around the other portion if so tightened.

The suture material is referred to as being “straightenable under applied tension”. This phrase is used to indicate that the suture material straightens sufficiently when drawn though tissue that the resilient memory does not interfere with sewing, or with insertion of the suture material through a trocar (typically with a channel of 5 or 10 millimeter diameter). It will be clear that the suture material need not become truly “straight” under these conditions, but is partially straightened in the sense that the radius of curvature of the coils typically increases.

The suture material is also referred to as “returning to its coiled state when released”. This requires that whatever straightening occurs during drawing of the suture material through the tissue is a primarily elastic deformation, and that the resilience of the suture material is sufficient to overcome the weight of suture material itself and return to its coiled state.

The term “knot” is used in its normal sense to refer to any tying together of two portions of thread-like material, in this case, suture material. Typically, the knots of the present invention are permanent knots formed from a sequence of twisting together the two portions and pulling them tight. Each such stage is referred to as a “throw”. Most preferably, at least the first throw of each knot includes a double-twisting of the portions together, and a total of at least two or more preferably at least three throws are used. However, variations of such knots, and other types of knots, also fall within the scope of the present invention. Particularly in a sequence of running stitches, knots which individually might not stand alone as permanent knots may be used.

The word “permanent” is used herein to refer to a knot of a type which, in durable suture material, would be expected to remain tied indefinitely under normal usage conditions. Clearly, in absorbable suture materials, the actual lifetime of a knot, even if “permanent” according to this definition, is limited by the diminishing tensile strength of the absorbable material over time.

Finally, the term “proximal” is used to refer to a position closer to tissue along the length of suture, while “distal” is used to refer to a position further from tissue along length of suture.

Turning now to the features of the present invention in more detail, needle 22 may be any type of needle known for use in sutures, chosen according to the procedure for which it is to be used and/or the surgeon's preferences. Needle 22 is preferably integrated with suture material 24, as is known in the art, so that the suture material follows as an extension of the needle so that it follows the needle through tissue without snagging or catching on the tissue. Techniques for attachment of the suture material to the needle are chosen according to the type of suture material used, and are well known in the art.

As mentioned above, the invention is essentially applicable to all available types of suture material. Certain types of suture material, and particularly synthetic polymer materials, have an inherent tendency to exhibit an elastic memory and/or can be engineered by varying proportions of components in co-polymer mixes to provide a desired stiffness. This elastic memory or stiffness has heretofore been widely considered a shortcoming of such materials for suture applications in that they tend to adopt the zigzag configurations typically used for packaging sutures prior to use. In the context of the present invention, the inherent elastic memory is preferably used to advantage to provide the desired biasing to the coiled state. The elastic memory can be appropriately set to the desired coiled state either by suitable winding of the suture material onto a forming template or roll during the manufacturing process or by an additional setting process. In some cases, a heat-setting process, performed separately. Heat-setting processes may also be used with certain natural suture materials.

In cases where inherent resilient memory of the desired suture is insufficient, or no significant resilient memory is exhibited, the present invention provides a number of techniques which may be used to provide the required resilient bias to a coiled state. Specifically, according to a first set of options, one or more strand of a springy material may be included within the suture material. In the case of a multi-strand wound or braided material, the springy material may be incorporated as part or all of one or more of the strands making up the braided material. Alternatively, either for braided or non-braided suture materials, the springy material may be implemented as part of a core (central or off-center) or an elongated rib passing along the length of the suture material. In the case of a core or rib, any suitable manufacturing technique may be used. For example, the suture may be formed around the core, or the core may be inserted into the suture material after is it formed, all according to the design considerations relevant to suture manufacture of each given type of suture material, as will be clear to one ordinarily skilled in the art.

The aforementioned springy strand implementations may employ any biocompatible material with suitable resilient properties. For non-absorbable sutures, examples include, but are not limited to, various metallic spring materials such as stainless steel and superelastic alloys such as nitinol. For bioabsorbable suture materials, the springy strand may be a relatively stiff strand of a suitably chosen synthetic bioabsorbable polymer. In each case, for a given type of resilient strand, the desired resilient properties (e.g., spring strength) may be ensured by suitable choice of the gauge of the material used.

According to a second set of options for generating the desired resilient bias to a coiled form, the coiled form is set by application of a suitable coating on the surface of the suture material. Examples of suitable coatings include, but are not limited to, poly-lactic co-glycolic acids, as well as simple biodegradable thin coats of shellac, varnish or starch. Depending upon the nature of the coating and of the underlying suture material, the coating may be applied by various techniques, including but not limited to dipping and spraying, while the suture material is held in a desired coiled form (e.g., wound around a forming template), of the coating may be applied in a non-resilient form and the suture material subsequently formed into the desired coiled shape and set by drying or heat treatment, all as appropriate to the type of coating. Optionally, in certain cases, it may be advantageous to configure the coating to be a rub-off coating which is at least partly destroyed during tightening of a knot, thereby avoiding possible impact of the resilient bias on security of the knot. In most cases, however, the relatively weak resilient bias of the suture material is not problematic, and the normal knots employed by surgeons are effective to permanently secure the suture despite the resilient bias.

It should be noted that the invention is not limited to the above-described options for generating the resilient bias of the suture material to its coiled form, and that any other available technique producing the required result also falls within the broad scope of the present invention as claimed. By way of additional non-limiting examples, the coiled form may in some cases be generated by mechanical techniques, such as by shaving, abrading or otherwise mechanically processing one side of the suture material to generate a tendency to coil up. In other cases, localized heating of the material along one side may be used to induce coiling or curling of the suture material. Alternatively, a coating may be applied selectively along one side of the suture material to cause the material to curl towards, or away from, the coated side.

Referring now again to FIGS. 4A-4C, this shows a first preferred mode of use of suture 20, corresponding to an implementation of the method of the present invention. After drawing a major part of suture 20 through tissue 26a, 26b as shown in FIGS. 4A and 4B, gripping tool 28 is inserted through one or more loops of leading portion 24a of the suture material from proximal to distal, i.e., in a direction away from tissue 26b, and the tool is then turned to grab part of trailing portion 24b, or trailing portion 24b is brought into the grip of gripping tool 28 and gripped. For at least the first throw of a surgical knot, at least two loops are preferably used. Trailing portion 24b can then be drawn back through the loops of leading portion 24a and pulled tight (using a second gripping tool, not shown). This procedure is then typically repeated for one or more additional throws in order to form the desired knot. For the second and subsequent throws of a knot, passing through a single loop may often be sufficient,

Use of the coils formed in the leading portion 24a of the suture material as described above is believed to have certain advantages. Specifically, the entire knotting process requires little or no motion of needle 22 which may be held separately, somewhat removed from the region of the tying. Nevertheless, it should be appreciated that knotting procedures according to the present invention may also be performed by passing leading portion 24a through at least one coil formed in trailing portion 24b. Thus, for example, FIG. 4D shows gripping tool 28 being inserted through at least one resiliently formed coil formed in the trailing portion 24b of the suture material from proximal to distal, which is then used to draw needle 22 back through the resiliently formed coils. As a further option, illustrated in FIG. 4E, needle 22 itself may be used directly to thread leading portion 24a through loops formed in trailing portion 24b by passing it from one gripping tool to another in a motion similar to the stitching process.

Although described herein primarily in a preferred case where the entirety of the leading or trailing portion (except of course for any part between the tissue and the knot) is passed through coils formed in the other portion, it should be noted that this is not necessarily the case. For example, certain knots may be formed by passing a folded over loop-portion of one of the portions through one or more resiliently formed coils in the other portion, and then threading an end of the suture material through that loop.

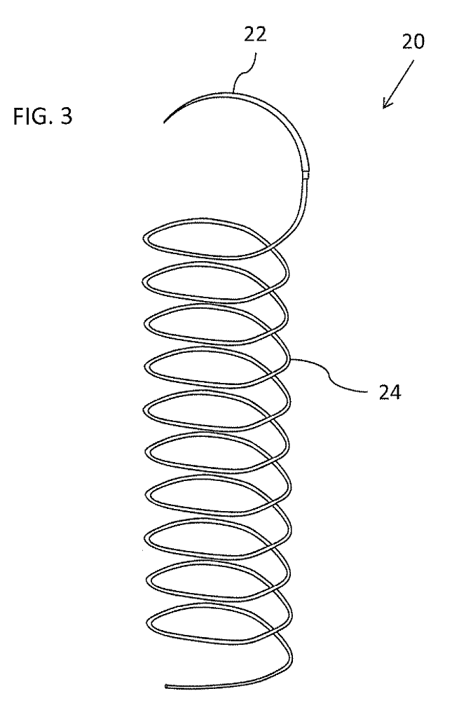

A further particular advantage of certain implementations of the present invention is that a single suture may be used to form a plurality of stitches. Given the awkward gross motions required to perform tying of sutures according to existing techniques (described above with reference to FIG. 2), it is normally considered necessary to sever the needle from the suture before tying. As a result, each suture is only used for a single stitch. Standard lengths of sutures for intra-corporeal stitches are typically around 11 centimeters which, due to the awkward procedure for forming a knot, is generally considered the minimum length suitable for forming and tying a single stitch. In contrast, according to the present invention, the motion required for tying is greatly reduced, and tying can be achieved safely and effectively with a relatively short trailing portion 24b and without detaching needle 22. As a result, it may be possible to tie a single stitch employing a significantly shorter suture of between 5 and 8 centimeters length. Alternatively, where a number of stitches are required, it becomes practical to sever the remainder of leading portion 24a from the knot after completing a first stitch and then to repeat the process for forming one or more additional stitches using the remainder of the same suture. Due to the increased efficiency of the knotting process, it is typically possible to tie more than one stitch from a standard 11 centimeter suture. Where more than two separate stitches are to be tied, an initial length of the suture material 24 significantly greater than the standard 11 centimeters may be used, for example, of at least about 15 centimeters length, and in certain cases in the range of 20-30 centimeters length. For continuous suturing from an initial anchoring knot to a finishing knot, lengths of 30-60 centimeters are preferably used. Despite the increased length, the resiliently formed coiled shape of the suture material ensures that it does not hang excessively below the needle when grasped. The entire length of suture material, when hanging freely from the needle, preferably extends no more than about 15 centimeters below the needle.

It should be noted that the present invention is applicable to any and all types of surgery, whether superficial surgery, open surgery or minimally invasive surgery. Even in superficial or open surgery, the presence of resiliently formed coils in the suture material greatly facilitates and accelerates the process of tying the suture. This advantage becomes much more sharply marked in minimally invasive procedures where forming of a knot is performed within a body cavity by manipulation of tools held outside the body, with the accompanying complications and difficulties, as discussed above with reference to FIG. 2. Similarly, in robotic surgery, where the tools used are robotically controlled, the present invention provides a profound advantage by circumventing the difficult manipulations required for winding part of the suture material around one of the tools.

Suture 20 as illustrated in FIG. 3 shows a major part, if not the entirety, of the length of suture material 24 as being resiliently biased to a helically coiled state. This option may be preferred for various reasons, such as simplicity of manufacture and convenience of packaging. However, it should be appreciated that a wide range of other coiled forms may be used which may vary considerably from a simple helical form while maintaining the underlying principles of the present invention. Thus, for example, the coils may have varying diameters (e.g., conical or other forms), may be individually spaced apart or spaced apart in groups, and may have coils with differing handedness and/or orientation. Two additional non-limiting examples will now be discussed briefly with reference to FIGS. 5 and 6.

Specifically, FIG. 5 illustrates a suture 30, constructed and operative according to the teachings of the present invention, in which the suture material includes a series of separated groups of resiliently biased coils 32a, 32b interconnected by connecting portions 34. In the example illustrated here, each group includes roughly three coils, although fewer or more coils, and non-whole numbers of loops, may also be used. Connecting portions 34 may be resiliently biased to a straightened or otherwise predefined form, or may have less tendency to return to any specific form. According to one particularly preferred option illustrated here, the handedness of the helical shape varies between groups of coils. In the case shown, groups 32a have a right-handed helix while groups 32b have a left-handed helix. The use of alternating handedness ensures that any tendency to cause a net twisting effect of the suture material when stretched straight or released cancels out between groups of coils. Parenthetically, both in this case and other examples of sutures illustrated herein, the illustration is not to scale, and the suture material typically extends significantly beyond the length and number of coils illustrated.

Turning finally to FIG. 6, this illustrates a further option for a suture 40 according to which groups of coils 42 are arranged in a plane roughly parallel to the extensional direction of the suture material. Here too, the groups of coils 42 are interconnected via connecting portions 44 which may have properties similar to, or differing from, the resiliently formed coils. In all other respects, the structure and function of sutures 30 and 40 may be understood by reference to the description of suture 20 above.

In each of the above cases, the resiliently formed coils preferably have an external diameter of between about 3 and about 30 millimeters, and most preferably between about 10 and about 20 millimeters. These dimensions are sufficiently large to allow insertion of a gripping tool through the coils while avoiding excess length of suture material to be pulled tight during tightening of each throw of the knots. Diameters of no more than about 20 millimeters are also preferred for intra-corporeal knot tying since the entire loops are more likely to be visible within the field of view of an endoscope.

It will be appreciated that the above descriptions are intended only to serve as examples, and that many other embodiments are possible within the scope of the present invention as defined in the appended claims.

What is claimed is:

1. A method for suturing tissue comprising:
(a) providing a length of suture material having a leading end and a trailing end, at least part of said length being resiliently biased to a coiled state, said at least part of said length being straightenable under tension and returning to said coiled state when released;
(b) drawing said leading end through or around at least one region of tissue so that a leading portion of the suture material lies between the tissue and said leading end and a trailing portion of the suture material lies between the tissue and said trailing end; and
(c) forming a knot in said suture material, said forming a knot including at least one sequence of:
(i) passing at least part of one of said leading and trailing portions through at least one resiliently-formed coil formed in the other of said leading and trailing portions, and
(ii) pulling tight said leading and trailing portions.

2. The method of claim 1, wherein said passing is performed by inserting a gripping tool through said at least one resiliently formed coil, gripping said other of said leading and trailing portions, and drawing said other of said leading and trailing portions back through said at least one resiliently formed coil.

3. The method of claim 2, wherein said gripping tool is inserted through said at least one resiliently formed coil from proximal to distal, and said drawing back through said at least one resiliently formed coil is from distal to proximal.

4. The method of claim 1, wherein said passing is performed so as to pass the entirety of said one of said leading and trailing portions through said at least one resiliently-formed coil.

5. The method of claim 1, wherein said passing is performed so as to pass said at least part of said one of said leading and trailing portions through at least two resiliently-formed coils formed in said other of said leading and trailing portions.

6. The method of claim 1, wherein said passing is performed so as to pass said trailing portion through said at least one resiliently-formed coil formed in said leading portion.

7. The method of claim 1, wherein said sequence of passing and pulling tight is performed at least three times.

8. The method of claim 1, further comprising severing a remainder of said leading portion from said knot and repeating steps (a) through (c) to form a subsequent knot.

9. The method of claim 1, wherein said leading end of said suture material is fixed to, and aligned as a continuation of, a needle.

10. The method of claim 1, wherein a major part of said length of said suture material is resiliently biased to said coiled state.

11. The method of claim 1, wherein said coiled state approximates to a helical form.

12. The method of claim 1, wherein said drawing and forming a knot are performed within a body cavity by manipulation of tools held outside the body.

13. The method of claim 1, wherein said drawing and forming a knot are performed by robotically controlled tools.

14. A suture for facilitating tying of knots, the suture comprising:
(a) a needle; and
(b) a length of suture material fixed to said needle,
wherein at least part of said length of suture material is resiliently biased to a coiled state, said at least part of said length being straightenable under tension and returning to said coiled state when released.

15. The suture of claim 14, wherein a major part of said length of said suture material is resiliently biased to said coiled state.

16. The suture of claim 14, wherein said coiled state approximates to a helical form.

17. The suture of claim 14, wherein said coiled state includes a plurality of coils having an external diameter of between about 3 and about 30 millimeters.

18. The suture of claim 14, wherein said coiled state includes a plurality of coils having an external diameter of between about 10 and about 20 millimeters.

19. The suture of claim 14, wherein said suture material is a biodegradable material.

20. The suture of claim 14, wherein said suture material is a braided material including at least one resilient strand configured to provide said resilient bias.

21. The suture of claim 14, wherein said suture material includes a resilient core configured to provide said resilient bias.

22. The suture of claim 14, wherein said suture material has a coating configured to provide said resilient bias.

23. A method for producing a suture for facilitating tying of knots, the method comprising:
(a) forming a length of material into a coiled form including a plurality of coils; and
(b) processing said length of material so as to impart to said length of material an elastic memory such that said length of material is resiliently biased to said coiled state, being straightenable under applied tension and returning to said coiled state when released.

24. The method of claim 23, wherein said processing includes heat treating said length of material.

25. The method of claim 23, wherein said processing includes applying a coating to said length of material.

26. The method of claim 23, further comprising fixing a needle at one end of said length of material.
